SEQUENCE LISTING (11 sequences)

SEQ ID NO: 1 - DNA, 6046 bp, Human Bcl-2 transfected neurons
gaattgctagcaattgctagcaattgctagcaattcataccagatcaccgaaaactgtcctccaaatgtgtccccctcacactcccaaattcgcgggcttctgcctcttagaccactctaccctattccccacactcaccggagccaaagccgcggcccttccgtttctttgcttttgaaagaccccacccgtaggtggcaagctagcttaagtaacgccactttgcaaggcatggaaaaatacataactgagaatagaaaagttcagatcaaggtcaggaacaaagaaacagctgaataccaaacaggatatctgtggtaagcggttcctgccccggctcagggccaagaacagatgagacagctgagtgatgggccaaacaggatatctgtggtaagcagttcctgccccggctcggggccaagaacagatggtccccagatgcggtccagccctcagcagtttctagtgaatcatcagatgtttccagggtgccccaaggacctgaaaatgaccctgtaccttatttgaactaaccaatcagttcgcttctcgcttctgttcgcgcgcttccgctctccgagctcaataaaagagcccacaacccctcactcggcgcgccagtcttccgatagactgcgtcgcccgggtacccgtattcccaataaagcctcttgctgtttgcatccgaatcgtggtctcgctgttccttgggagggtctcctctgagtgattgactacccacgacgggggtctttcatttgggggctcgtccgggatttggagacccctgcccagggaccaccgacccaccaccgggaggtaagctggccagcaacttatctgtgtctgtccgattgtctagtgtctatgtttgatgttatgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcgttaacagatctgtcgactaatacgactcactatagggcgaattaattccggttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaagcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataataactcgaggatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 2 - DNA, 20 bp, Human Bcl-2 transfected neurons
tgccattggctaggtgcacg

SEQ ID NO: 3 - DNA, 20 bp, Human Bcl-2 transfected neurons
gaacctgcgtgcaatccatc

SEQ ID NO: 4 - DNA, 29 bp, Human Bcl-2 transfected neurons
aaaggatccgccttgtagaagcgcgtatg

SEQ ID NO: 5 - DNA, 27 bp, Human Bcl-2 transfected neurons
aaaggatccttccgtgtttcagttagc

SEQ ID NO: 6 - DNA, 20 bp, Human Bcl-2 transfected neurons
ccacggggacgtggttttcc

SEQ ID NO: 7 - DNA, 19 bp, Human Bcl-2 transfected neurons
ggtcgatgtgtctgtcctc

SEQ ID NO: 8 - DNA, 30 bp, Human Bcl-2 transfected neurons
aaaagcggccgctttccacaccctaactga

SEQ ID NO: 9 - DNA, 18 bp, Human Bcl-2 transfected neurons
cagataacaatgggcatg

SEQ ID NO: 10 - DNA, 28 bp, Human Bcl-2 transfected neurons
aatagatctcagccctcactccttctct

SEQ ID NO: 11 - DNA, 28 bp, Human Bcl-2 transfected neurons
aatagatcttttccacaccctaactgac

(19) United States
(12) Patent Application Publication
(10) Pub. No.: US 2004/0001808 A1
(43) Pub. Date: Jan. 1, 2004

(54) Compositions and methods for the use of Bcl-2 transfected neurons

(76) Inventors: Kenneth Haglid, Hovas (SE); Shu Wang, Singapore (SG)

Correspondence Address: KRAMER LEVIN NAFTALIS & FRANKEL LLP, INTELLECTUAL PROPERTY DEPARTMENT, 919 THIRD AVENUE, NEW YORK, NY, 10022, US

(21) Appl. No.: 10/164,965

(22) Filed: Jun. 7, 2002

(51) Int. Cl. A61K 48/00; C12N 5/08; A61K 31/203

(52) U.S. Cl. 424/93.21; 435/368; 514/559

(57) ABSTRACT

The use of Bcl-2 transfected neurons and/or stem cells for the production of a pharmaceutical preparation for in vivo treatment of neurological conditions such as Parkinson's disease is disclosed. Also disclosed is a method for treatment of neurological conditions, wherein Bcl-2 transfected neurons are transplanted to a patient.

### FIELD OF THE INVENTION

The present invention relates to compositions and methods for treating neurological conditions.

### BACKGROUND OF THE INVENTION

Neurological disease and injury affect millions of people in the U.S. and abroad. The cost in treating these patients is many billions of dollars annually. In fact, according to the Parkinson's Foundation as many as 1.5 million people suffer from Parkinson's disease in the U.S. alone. About 50,000 Americans are diagnosed with Parkinson's each year and the total cost of health care for Parkinson's patients exceeds $5 billion annually. Other countries have even higher rates of Parkinson's disease. The incidence and associated costs of other neurological conditions are similarly severe.

Strategies of treatment of neurological disease or injury have been largely unsuccessful. For example, the use of a variety of pharmaceutical preparations for the treatment of Parkinson's disease has done little to provide long-term relief to these patients. Standard pharmaceutical treatments eventually result in ineffective treatment of symptoms and/or unacceptable side effects. Moreover, these traditional treatments are intended purely to alleviate symptoms rather than to resolve the underlying disease or injury. Treatment of other neurological conditions is similarly symptom oriented.

The transplantation of various cells types of the central nervous system (CNS) (i.e. neurons, astrocytes or oligodendrocytes) as well as Schwann cells of the peripheral nervous system (PNS) offers possibilities to reduce symptoms or even cure diseases such as Alzheimer's disease, Parkinson's disease or amyotrophic lateral sclerosis (ALS), in which a proportion of these cells are lost or malfunctioning.

However, strategies for treatment of CNS disease and injury so far have been largely unsuccessful. Previously described CNS transplants die rather rapidly and/or lose the desired function. Human fetal mesencephalic tissue grafts do not survive long enough to ameliorate Parkinson-like symptoms in drug-induced disease or in neuro-degenerative disorders. Likewise, immortalization of CNS cells using constructs containing temperature-sensitive promoters to aid in transplantation of genetically engineered precursor cells of glia or neurons, have been largely unsuccessful e.g., glia-neuron mixed transplant (Cattaneo, E., and R. McKay 1991 TINS 14.338-340, Refranz, P. J., et al. 1991 Cell 66:713-729). Another strategy has been the use of transformed neuron-like cell lines from CNS tumors. However, neoplastic neurons cannot be kept outside the cell cycle permanently and usually develop new tumors (Fung, K. et al. 1992 J. Histochem. Cytochem. 40:1319-1328; Wiestler, O. D. et al. 1992 Brain Pathol. 2:47-59).

Accordingly, there is a tremendous need for new and useful compositions and methods for treating CNS disease and injury. More specifically there is a tremendous need for new and useful compositions and methods of transplanting neurons and other cells of the CNS and PNS to treat neurological diseases and injuries including Parkinson's disease, Alzheimer's disease, and amytrophic lateral sclerosis (ALS). In particular there is a need for compositions and methods for treating Parkinson's disease by transplanting dopamine-producing neurons into the CNS of mammals.

### SUMMARY OF THE INVENTION

The present invention provides compositions and methods for the treatment of neurological diseases and injuries in mammals, including humans. Such diseases and injuries include, but are not limited to Parkinson's disease, Alzheimer's disease, amytrophic lateral sclerosis (ALS) and spinal injury. In a preferred embodiment the disease is Parkinson's disease.

An object of the present invention is to provide Bcl-2-transfected neurons which may be included in pharmaceutical preparations for the treatment of neurological dieases and injuries in mammals, including humans.

A further object of the invention is to provide Bcl-2-transfected neurons which may be formulated into compositions for transplantation into the CNS and/or PNS of a mammal to treat neurological diseases or injuries in mammals, including humans.

An additional object of the present invention is to provide Bcl-2-transfected neurons which may be formulated into compositions for transplantation into the CNS of mammals, including humans, for the treatment of Parkinson's disease. In a preferred embodiment the Bcl-transfected neurons produce dopamine and alleviate the symptoms of Parkinson's disease upon transplantion.

A further object of the present invention is to provide Bcl-2-transfected NT2N neurons and in particular neurons from a genetically engineered neuronal cell line of human origin comprising Bcl-2, suitable for transplantation into mammals including humans, in need of such treatment.

### BRIEF DESCRIPTION OF THE DRAWINGS

FIG. 1 shows a Western blot of equal amounts of total protein for the Bcl-2-protein. Parental cells (pNT2) and differentiated neurons, transfected with the empty vector (NT2Nneo) or with the human Bcl-2 gene. Bcl-2-C5, Bcl-2 A5 or Bcl-2-A4 denote isolated and successfully transfected clones.

FIG. 2 shows the results after administration with a stereotactic device of 1×10⁵ NT2Nneo neurons (the two pictures to the left) or the same number of Bcl-2-neurons (clone Bcl-2-C5) (the two pictures to the right) into the hippocampus of Sprague Dawley rats.

FIG. 3A shows the results of transplantation of 1×10⁵ Bcl-2-neurons to the right striatum at 4 weeks and the visualization is as shown in FIG. 2. FIG. 3B is a magnified view of a portion of FIG. 3A.

FIG. 4A shows the transection site in the spinal cord of Sprague Dawley rats at the Th IX-Th X level. FIG. 4B shows the regeneration of the human Bcl-2 nerve cells in transected rat spinal cords. FIG. 4C shows 4 photomicrographs of slides from the transected rat spinal cords. In the microscopic photomicrographs A-D, A shows human Bcl-2 neurons (black) with regenerating axons directed to the right. The white dots indicate where the photomicrographs were obtained. Photomicrograph D is a magnification of the regenerated neurons in B.

FIG. 5 illustrates the enzymatic pathway for converting tyrosin into dopamine.

FIG. 6 provides the DNA sequence of clone pLXSN-IRES-hTH-1.

### DETAILED DESCRIPTION OF THE INVENTION

When appearing herein, the following terms shall have the definitions set forth below.

The term “patient” used herein relates to any human or non-human mammal in need of treatment according to the invention.

The term “treatment” used herein refers both to treatment in order to cure or to treatment in order to improve a disease or condition.

The term “condition” may refer to disease, syndrome, disorder, malfunction or injury.

The present invention provides compositions and methods for the treatment of neurological conditions in patients. Such conditions include, but are not limited to, Parkinson's disease, Alzheimer's disease, amytrophic lateral sclerosis (ALS) and spinal injury. In a preferred embodiment the disease is Parkinson's disease.

The present invention also provides Bcl-transfected neurons which may be included in pharmaceutical preparations for the treatment of neurological dieases and injuries in mammals, including humans. The present invention further provides Bcl-transfected neurons which may be formulated into compositions which may be transplanted into the CNS and/or PNS of patients to treat neurological conditions. Bcl-transfected neurons are resistant to apoptosis and after in vitro differentiation, these cells unexpectedly survive in vivo following transplantation for at least 6 to 8 months in a mammal model. Importantly, these cells do not rely on proliferation to survive. For example, Bcl-2 transfected neurons transplanted to a transected rat spinal cord are capable of in vivo survival and regeneration for more than 180 days.

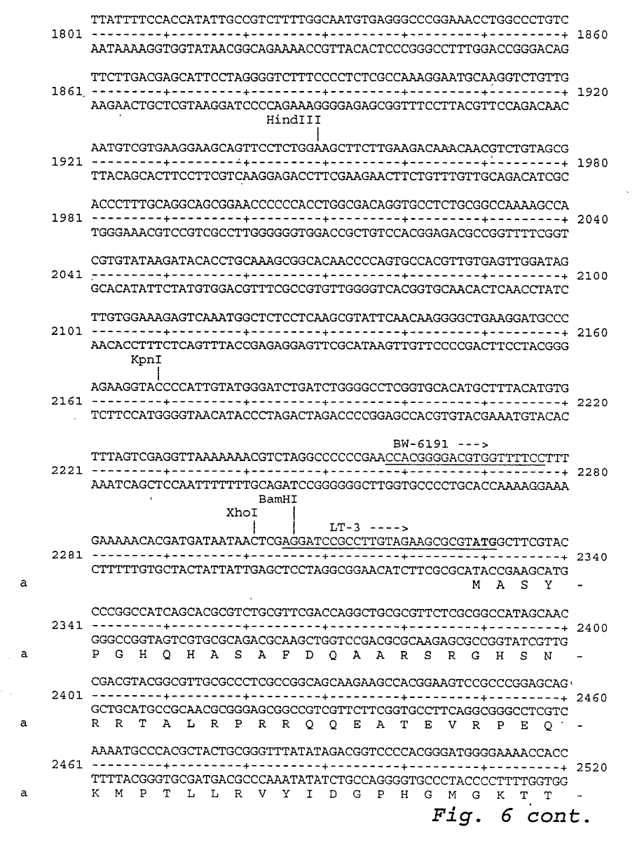

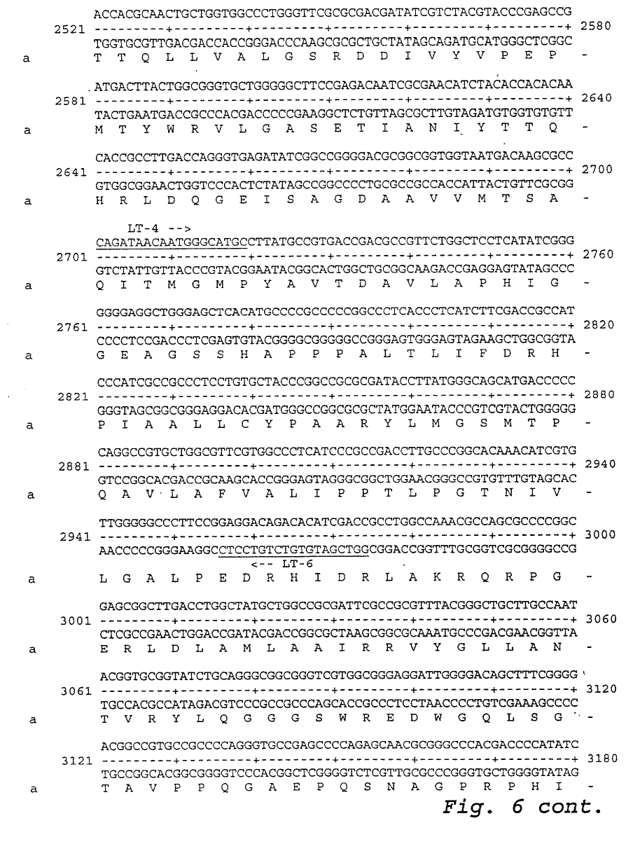

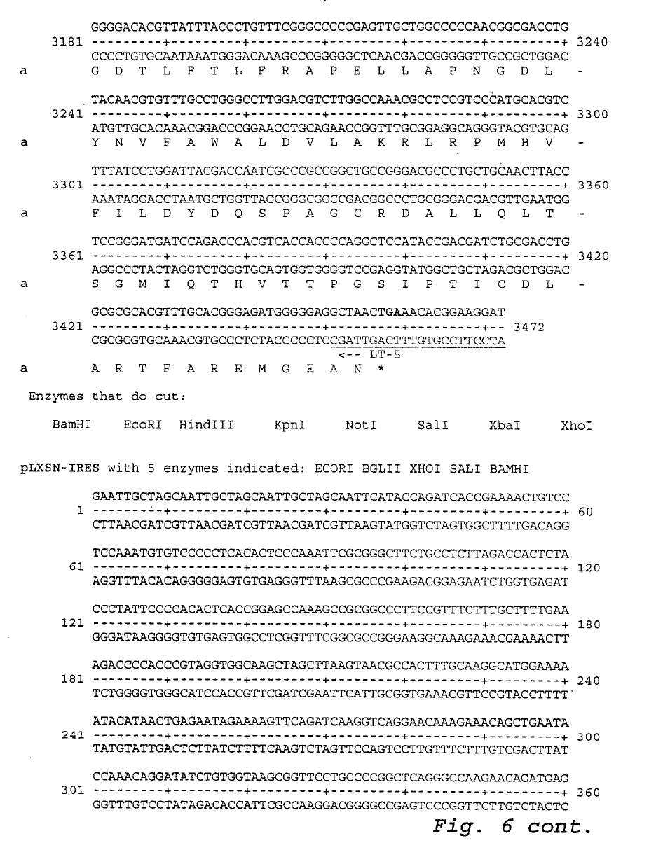

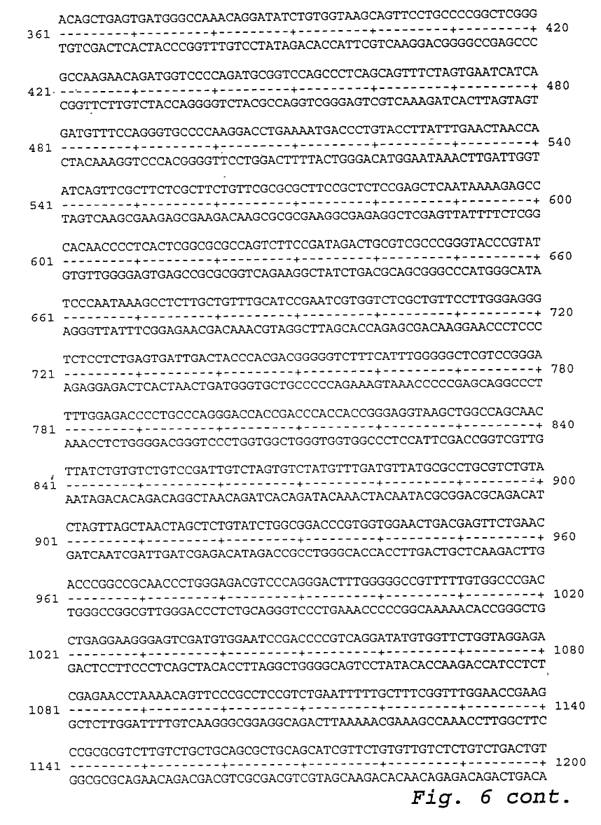

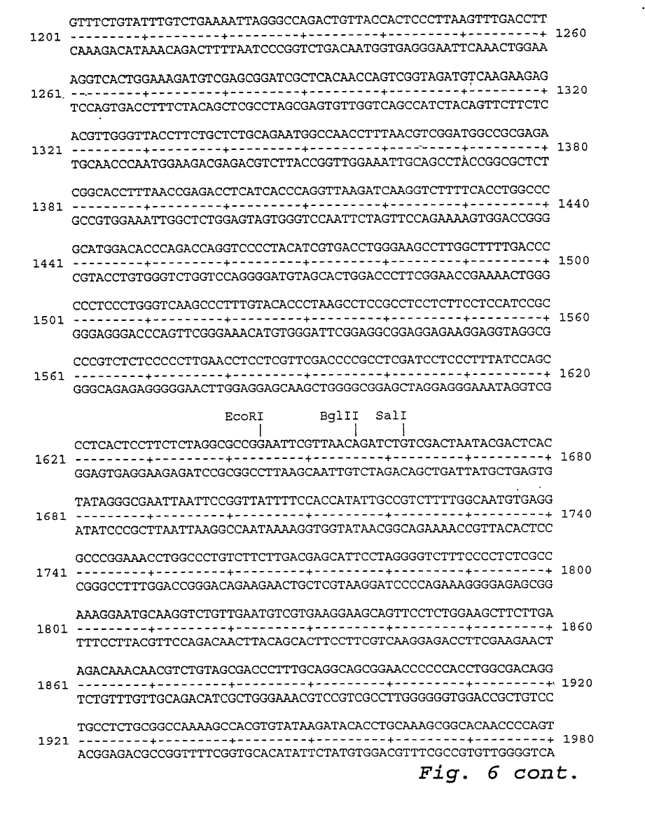

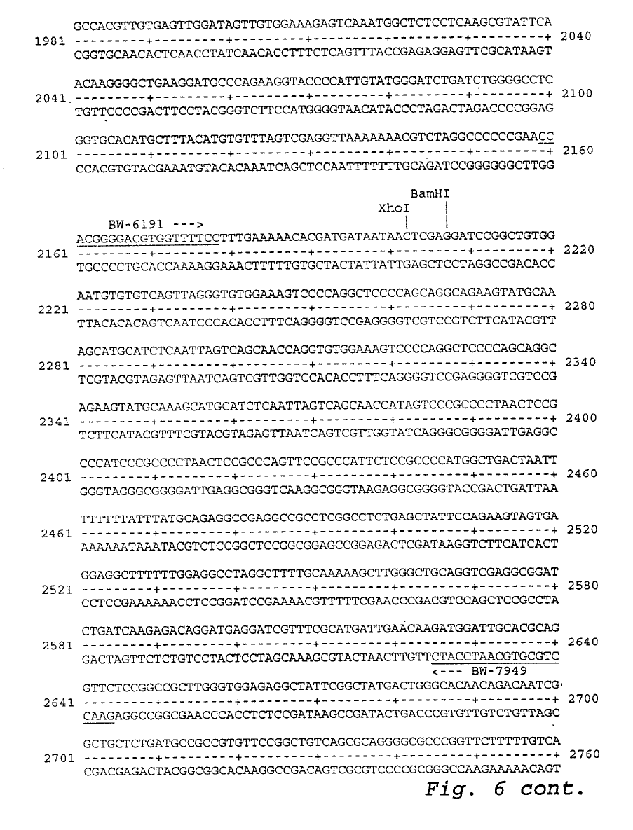

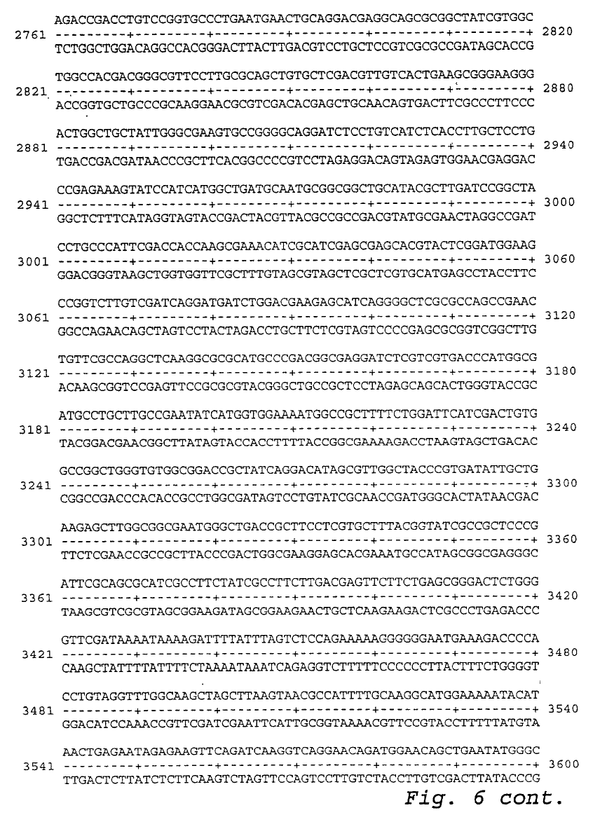

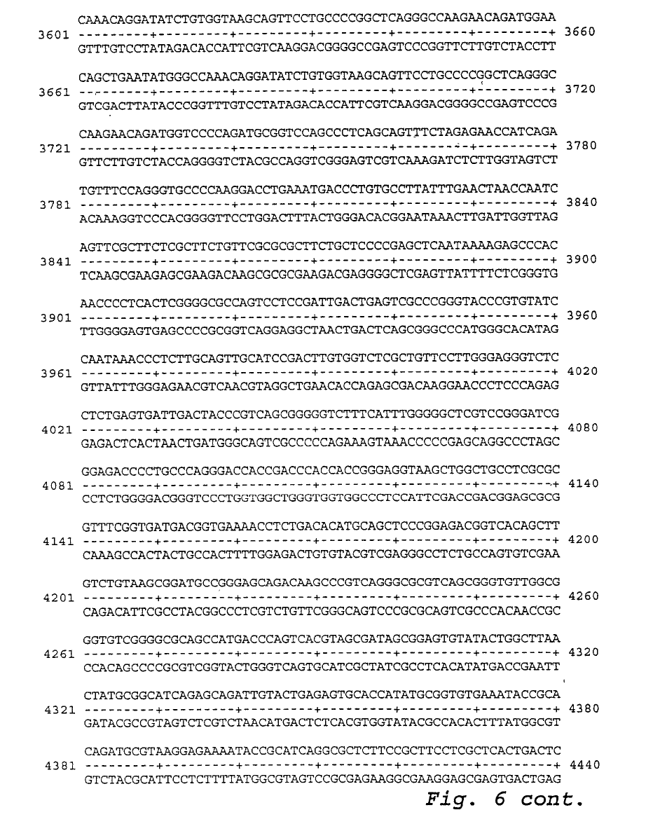

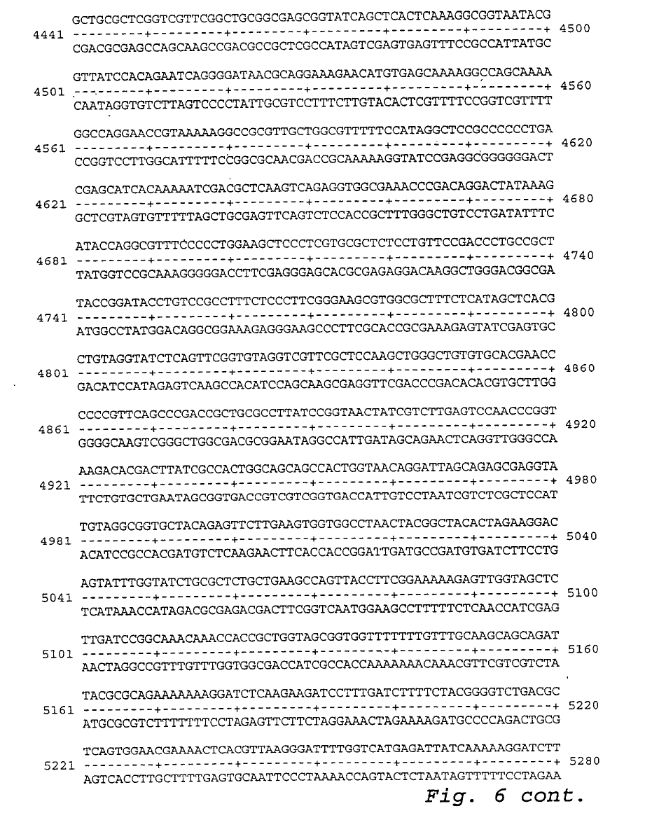

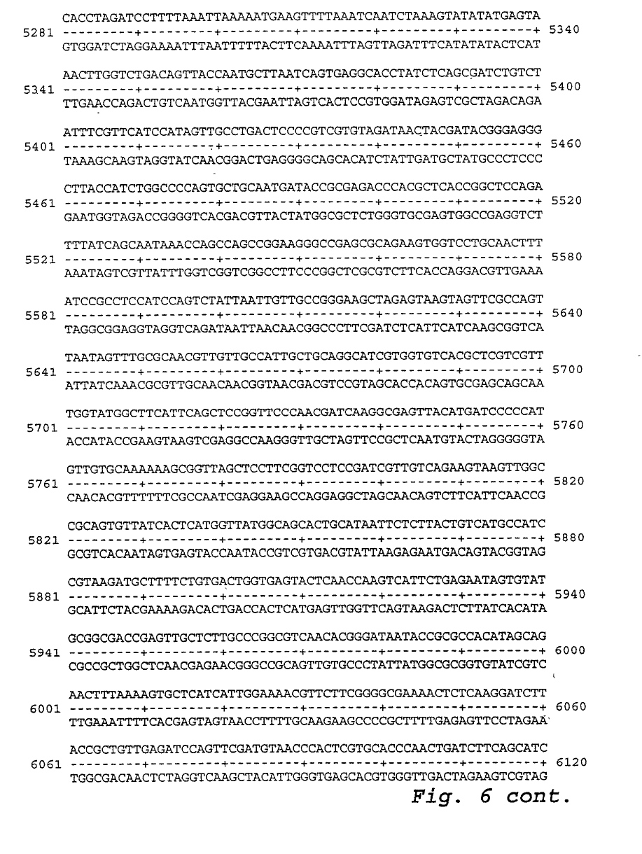

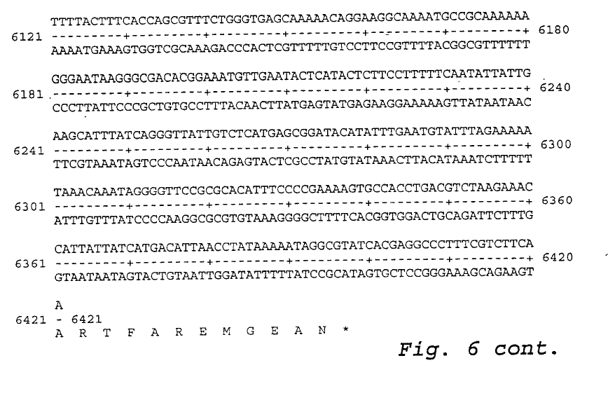

The invention futher provides Bcl-2-transfected neurons which may be formulated into compositions which may be transplanted into the CNS of mammals, including humans, for the treatment of Parkinson's disease. In a preferred embodiment the Bcl-2-transfected neurons produce dopamine and alleviate the symptoms of Parkinson's disease upon transplantion. In another preferred embodiment cloned cells are provided which are capable of being transplanted into a patient to produce dopamine. A preferred clone is designated as pLXSN-HSVTK-IRES-hTH-1 and corresponds to Seq. ID No. 1 and is shown in FIG. 6. The present invention also provides clones having substantial homology to Seq. ID No. 1. Transplanted, dopamine-producing Bcl-2-transfected neurons are capable of surviving in vivo for at least 240 days.

The present invention also provides Bcl-2 transfected neurons a preferred embodiment of which specifically provides Bcl-2 transfected NT2N neurons or stem cells, and in particular neurons from a genetically engineered neuronal cell line of human origin comprising Bcl-2, all of the above suitable for transplantation into the CNS and/or PNS of patients in need of such treatment. The above-referenced genetically engineered neuronal cell line of human origin has been deposited as accession No. DSM ACC2505 at DSMZ-Deutsche Sammlung von Mikroorganismen and Zellkulturen GmbH on Apr. 26, 2001.

Experimental Materials, Methods and Results

Cell Culture

NT2 cells, purchased from Stratagene Ltd. (Cambridge, UK) were cultured in Opti MEM-I (Life Technologies, Gaithersburg, Md.) medium supplemented with 5% (v/v) fetal calf serum (FCS) and penicillin/streptomycin (P/S) as previously described (Pleasure, S J. et al. J, Neuroscie. 12 (1992) 1802-1815).

Western Blotting

Cells were lysed in a small volume of 150 mM NaCl, 1% NP-40, 50 mM Tris, pH 8.0 by sonication. Protein concentrations of the cell lysates were determined in microtiter plates (Pierce, Rockford, Ill., USA). Equal amounts of protein extracts were resolved by 12% sodium dodecyl sulfate polyacrylamide gel electrophoresis (SDS-PAGE), and transferred to nitro-cellulose membranes. Equal loading of proteins from each cell lysate were verified by Coomassie blue staining of the transferred gels. The membranes were probed with specific monoclonal mouse anti-human bcl-2 antibody (1:1000, Clone 124, DAKO, Denmark). Horseradish peroxidase-conjugated rabbit anti-mouse immunoglobulin was used as the secondary antibody. Detection was performed with enhanced chemiluminescence (ECL, Amersham, England) according to the manufacturer's instructions.

Gene Transfection

Vectors generating the human Bcl-2 cDNA were constructed using standard recombinant DNA techniques (Sambrook, J., et al., 1989 Molecular Cloning: A Laboratory Manual. Cold Spring Harbor Press, New York). The plasmid pB4, harboring a 0.91 kb cDNA from human Bcl-2 gene, was obtained from the American Type Culture Collection (Rockville, Md.). The eukaryotic expression vector pcDNA3 was obtained from Invitrogen (Carlsbad, Calif.). An EcoRi cDNA with Bcl-2 cDNA from pB4 was subcloned into the pcDNA3. Restriction enzyme analysis and nucleic acid sequencing ascertained the orientation of the Bcl-2 inserts.

NT2 cells were transfected with the plasmid using the PerFect lipid transfection reagents (Invitrogen, Carlsbad, Calif.) according to the procedure recommended by the manufacturer. Briefly, the cells were seeded on six-well plates at a density 2×10⁵/well and incubated overnight. Immediately before transfection, the cells were washed three times in serum-free Opti-MEM 1 medium. Supercoiled plasmid DNA (2 μg/ml) and PerFect 1 (12 μg/ml) in the Opti-MEM 1 medium were mixed in polystyrene tubes and preincubated for 5 min at room temperature before being added to the cells. After incubation for 4 h at 37° C., the transfection medium was removed and Opti-MEM 1 medium containing 5% of FCS was added. Selection of stable clones was initiated 24 h after transfection with 0.4 mg/ml G418. Stable transfected, cloned cells were characterized by Western blot analysis and maintained in Opti-MEM 1 medium containing 5% FCS and 0.2 mg/ml of G418.

Construction of Bcl-2 Cells for the Production of Dopamine and Control of Cell Proliferation

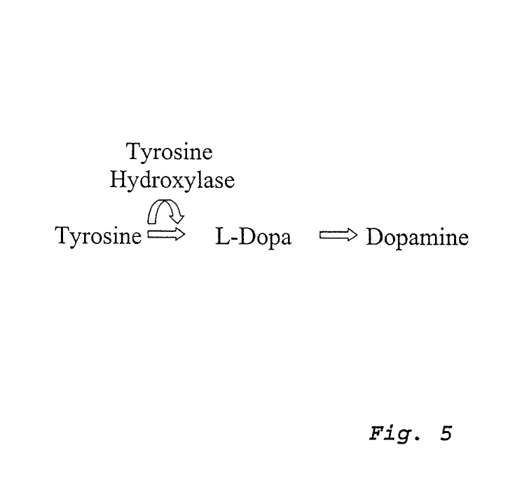

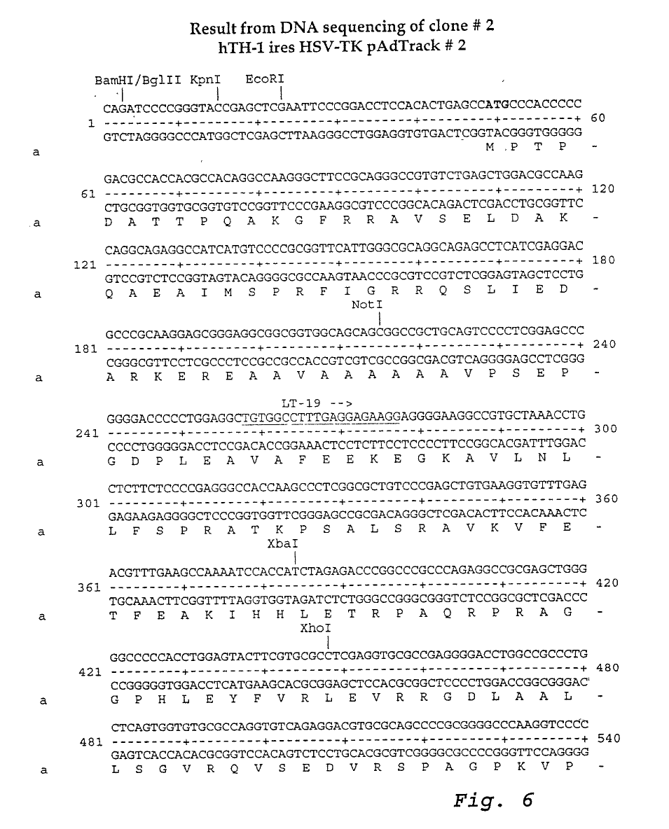

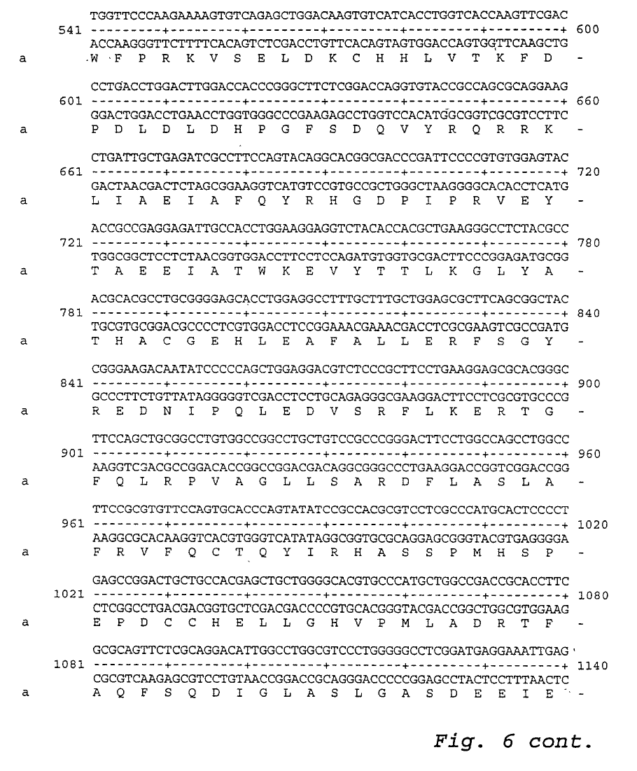

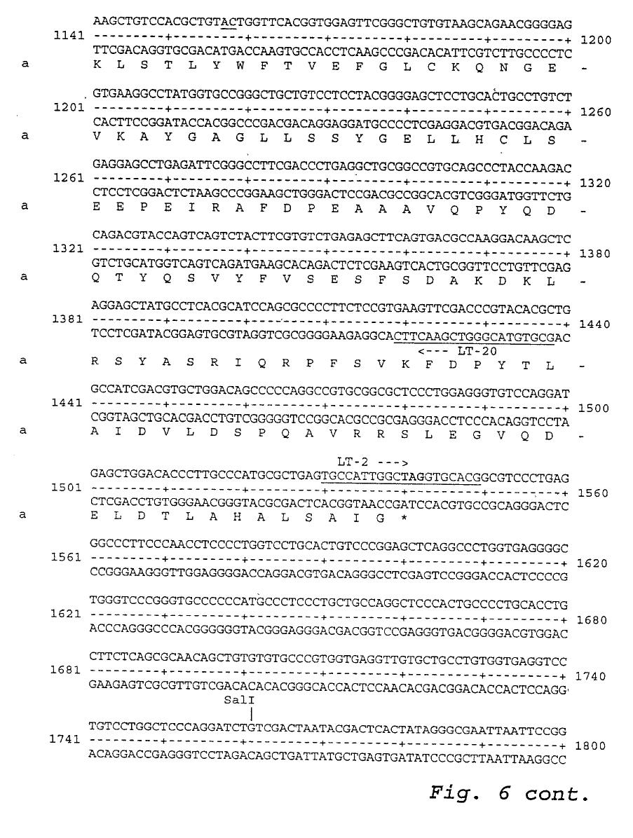

FIG. 5 illustrates the enzymatic pathway for converting tyrosin into dopamine. In the present invention, Bcl-2 transfected human neuronal cells were constructed as described below to produce dopamine in such a way that these cells can be transplanted in vivo to cure Parkinson's disease in patients. Differentiated cells can be killed by drugs that activates the thymidine kinase, such as the drug Ganciclovir, which is toxic for cells expressing the Herpes Simplex Thymidine Kinase (HSV-TK). Ganciclovir is a pro-drug that is activated via phosphorylation by HSV-TK. In this way cell proliferation can be controlled.

Human tyrosine hydroxylase 1 (hTH-1) was cloned under the control of a CMV promoter in a vector (pAd-Track-CMV). The gene for Herpes Simplex Virus thymidine kinase (HSV-TK) was also cloned in this vector. These two genes are both controlled by the same CMV promoter and the genes are separated by an Internal Ribosome Entry Site (IRES) so that the single transcript will be translated as two separate proteins. The gene for Green Fluorescent Protein (GFP) was also included in this same construct. The expression of the GFP gene is also regulated by a similar but not identical CMV promoter. Both the CMV promoters have about the same strength but are made non-identical to avoid recombination that can create problems when two identical elements are present.

If this construct is used for transfection of cells, it will make the cells express the green fluorescent protein and the cells expressing this protein can be sorted by, e.g., a FACS sorter. This will enable a fast selection for cells with a strong expression, which e.g. may be identified by the fact that the living cells expressing the GFP are green in a fluorescence microscope. The expression of the two other genes will be at a level similar to the expression of the GFP gene. This is advantageous if toxin selection is to be avoided. The expression of the green protein will make it possible to sort the cells by FACS. However, the GFP can be omitted from the construct and the construct can be co-transfected with a gene encoding proteins that make them resistant to cytotoxic drugs, such as hygromycin or puromycin, after which drug selection can be carried out.

The construct is designed for the Bcl-2 transfected neuronal cells. These cells express the neomycin resistance gene which make it possible to use both alternatives, i.e. hygromycin or puromycin.

The bcl-2 gene renders the cells resistant to apoptosis and after in vitro differentiation, these human cells can survive for at least 6 to 8 months in the brain of immunocompetent rats and the cells did not rely proliferation for survival.

Method for the Production of Cells Producing Dopamine

The human cDNA gene for tyrosine hydroxylase (hTH-l) was cloned in a bluescript vector pBS.KS(+). The cDNA fragment of hTH-1 was cut out from pBS.KS(+)hTH-1 by the restriction enzyme BamHI. This BamHI fragment was then ligated into the BglII-site of pLXSN-IRES (see sequence below). By means of PCR, using the primers LT-2 (Seq. ID No. 5)+BW-7949 (Seq. ID No. 3), the orientation of the fragment in the vector was controlled. The ends of the a clone with the correct orientation was checked by DNA sequencing. The correct clone was designated pLXSN-IRES-hTH-1 the sequence of which is shown in Seq. ID No. 1 and in FIG. 6.

Cloned thymidine kinase from herpes simplex virus (HSV-TK) was amplified by PCR, using the primers LT-3 (Seq. ID No. 6) and LT-5 (Seq. ID No. 8) which contain BamHI-sites. This amplification was performed with an enzyme with proofreading capacity (pfu-polymerase). The PCR-product was digested with BamHI and the resulting fragment was then ligated with BamHI digested pLXSN-IRES-hTH-1. Clones with correct orientation of the fragment was controlled by PCR with the primers BW-6191 (Seq. ID No. 2)+LT-6 (Seq. ID No. 9).

On a clone with the correct orientation of the HSVTK gene, DNA sequencing was performed. Sequencing was carried out using the primers BW-7962 (Seq. ID No. 4), BW-6191 LT-4 (Seq. ID No. 7) and LT-6. A proper clone was identified and was designated pLXSN-HSVTK-IRES-hTH-1.

The entire hTH-1/IRES/HSV-TK fragment was PCR amplified using primers LT-7961 (plx-Bgl2-5′) (Seq. ID No. 12) och LT-7962 (plx-Bgl2-3′) (Seq. ID No. 13). The amplified fragment was then digested with BglII and then ligated into the BglII site of pAdTrack-CMV.

The resulting clones were controlled by PCR and the whole insert was verified by DNA sequencing. The results of DNA sequencing of clone No. 2 is shown in FIG. 6.

Cells produced in the above-described manner produce dopamine and areuseful for in vivo treatment of conditions such as Parkinson's disease.

Neuronal Differentiation

In order to induce neuronal differentiation, the naive NT2 or Bcl-2 cells were cultured for 5 weeks with 1×10⁻⁵ M retinoic acid in DMEM (Life Technologies, Gaithersburg, Md.) supplemented with 10% FCS and P/S. The cells were then replated at a 1:6 split ratio. Two days later, the mitotic inhibitors (Sigma, St. Louis, Mo.) cytosine arabinoside (1 μM), fluorodeoxyuridine (10 μM) and uridine (10 μM) were added to the medium in order to inhibit the division of non-neuronal cells. Eight days later, the differentiated NT2N or Bcl-2 neurons were separated from non-neuronal cells. This was achieved with mechanical dislodgment of the cells by gentle striking of the flasks, subsequent to a 2 min treatment of the cultures with 0.05% trypsin-EDTA (Life Technologies, Gaithersburg, Md.). Purified NT2N cells or Bcl-2 neuronal cells were plated on Matrigel (Collaborative Research, Bedford, Mass.) coated culture plates and maintained in DMEM with 10% FCS, P/S containing the mitotic inhibitors.

Neuronal Transplantation to the Rat Brain

1×10⁵ NT2N or Bc12-C5 neuronal cells were stereotactically injected under an operation microscope into the hippocampus or striatum of Sprague Dawley rats and cared for according to the Swedish government regulations. All procedures were approved by the Göteborg University animal care committee. Twenty-two rats were transplanted with Bcl-2 neurons and 22 rats with NT2N neurons. Briefly, male Sprague-Dawley rats (BW 250-300 g) were anesthetized with ketamine, ketalar (Parke-Davies-100 mg/kg) and xylazine (Rompun Vet-Bayer-5 mg/kg). The skin was cut in the midline to expose the scull and 4 holes were drilled bilateral from the midline through the scull by a micromotor drill. Striatum (caudate putamen) or hippocampus were injected on one side with NT2N cells and on the other with Bcl-2 neurons (1×10⁵ cells in both experiments), respectively. The stereotaxic co-ordinates were, for the striatum, 0.3 mm anterior from the bregma and 3.5 mm lateral from the central suture and at a depth of 5.0 mm ventral to the dura and for hippocampus: 4.4 mm posterior to bregma, 3.2 mm lateral to the central suture and 3.5 mm ventral to the dura. The anesthetized rats were then sacrificed at varying survival times and fixed as described below. Immunocytochemistry for the human NCAM (neuronal cell adhesion molecule) stained only human neurons and was consequently used as tracer for human cells in the rat.

Spinal Cord Transection and Transplantation

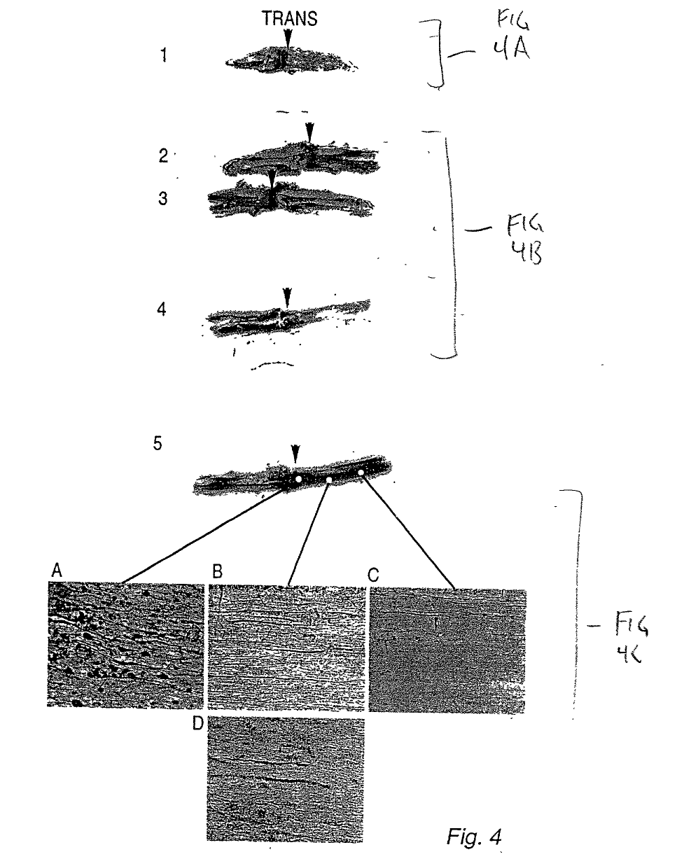

Adult female Sprague Dawley rats (n=16, 3 months old, 220 g) were housed and cared for according to the Swedish government regulations, and all procedures were approved by the Lund University animal care committee. Surgery was performed under completely aseptic conditions with a surgical microscope. Before each surgical procedure and perfusion, the rats were anesthetized by an intramuscular injection with a mixture of ketamine (62.5 mg/kg) and acepromazine. The surgical area was shaved and antiseptically prepared with 75% ethanol. The skin was opened dorsally, and the underlying adipose tissue and muscle layers were incised along the midline and kept apart with a tissue spreader to obtain a clear view of the Th IX-Th X vertebrae. After removal of the rostral part of the dorsal lamina of Th IX-Th X using a bone nibbler, the dura was opened and the spinal cord transected completely with microscissors. Hamilton syringes (Hamilton Company, Reno Nev., USA) were used to deliver an injection of either vehicle (PBS) or Bcl-2 neuronal cell suspension (3 μl containing approximately 200,000 cells). FIG. 4 shows the spinal cord transection sites. In order to minimize trauma to the cells a glass canula (0.2 mm external diameter) was mounted on the tip of a Hamilton syringe needle. Next, the syringe was held in a rat stereotactic apparatus and the tip of the glass canula was introduced 1.0 mm deep into the spinal cord lesion. After a 2-min waiting period, the content of the syringe was injected between the cut ends of the spinal cord over a 5-min period. The tip of the canula was left in place for another 5 min. It was then withdrawn. The muscles and skin were then closed with interrupted sutures (3, 0 silk, Ethicon Inc., Somerville, Mass.).

The rats were carefully nursed after the operation and were supplied with a catheter in the urinary bladder. Post-operative care included warming on a heating pad, administration of the painkiller Temgesic twice daily and evacuation of the bladder (for neurogenic bladder). Animals with neurological or health problems were euthanized. Antibiotic prophylaxis consisted of 2 ml penicillin intramuscularly immediately prior to surgery. Penicillin was then given once daily for 7 days.

Immunocytochemistry

Rat Brain

The rats were sacrificed and fixed by perfusion as described above. The brains or spinal cords were then postfixed in the same fixative over night and then equilibrated in 20% sucrose in phosphate buffered saline solution (PBS). 2.3-2.5 cm segments of the spinal cord, having the transplant in the middle (marked TRANS in FIG. 4) were embedded in geletin and freeze-sectioned in 25 μm thick serial sections containing the transplants. The segments were immunostained by free-floating with mouse monoclonal antibodies which only recognize the human neuronal cell adhesion moloecule (NCAM). The monoclonal antibodies stained only human neurons and were used as a tracer for human cells in the rat.

Free-floating sections were washed in TBS (25 mM Tris, 150 mM NaCl, 3 mM KC 1, adjusted to pH 8.0) and incubated in 0.6% H₂O₂ in TBS for 30 min to quench endogenous peroxidase activity. After two washes in TBS and a 30 min incubation in 5% normal horse serum (DAKO), they were incubated with the monoclonal antibodies against human NCAM diluted in 1% BSA-TTBS (TTBS=TBS containing 0.05% Tween-20) at 4° C. overnight. Consecutively, the sections were incubated with biotinylated horse anti-mouse-IgG diluted 1:200, followed by incubation with avidin-biotin-horseradish peroxidase complex, diluted 1:200. Then they were developed in GDN solution (glucose-diaminobenzidine-nickel, Yang et al., Appearance of neuronal S-100b during development of the rat brain, Dev. Brain Res., 91 [1996] 181-189. 1995a) for 5-20 min.

All procedures were carried out with gentle shaking. Stained sections were washed in TTBS, mounted on gelatine coated slides, dehydrated through a gradient of alcohol, cleared with xylene, and coverslipped with Mountex (Histolab, Göteborg, Sweden). Negative controls underwent the same procedures with omission of the primary antibodies.

NCAM immunoreactivity was only detectable in transplanted human cells. This was evaluated with a CH-2 Olympus microscope. Pictures were taken under Zeiss microscope and a Kodak TMAX-100 black-and-white film was used. The size of the transplants was estimated in an IBAS system (Leitz microscope equipped with a Microvid Image Analysis System). The maximal surface area stained with the monoclonal antibody against human NCAM was determined in serial sections of each transplant.

Spinal Cord Embedding and Sectioning

After the selected survival times, animals were again anesthetized and perfused transcardially with 50 ml ice cold PBS followed by 350 ml of 4% paraformaldehyde in PBS. When the spinal cord had been dissected, it was attached to a wax strip to prevent curvatures, immersion-fixed for 16 h and cryoprotected in 30% buffered sucrose for 24 hours, all at 4° C. The thoracic spinal segment including, 10 mm proximally and distally to site of the lesion, was removed and incubated overnight in a phosphate-buffered saline, 10% gelatine containing 0.06% sodium azide, to prevent bacterial and fungal growth, at 37° C. The spinal segments were then embedded in gelatine blocks that were hardened for 20 minutes at −20° C., and then immersion fixed in 4% paraformaldehyde (at least 4 hours) and cryoprotected in 30% sucrose (16 h), all at −4° C. Longitudinal (horizontal) 40 um sections were cut on a freezing sliding microtome, kept in anatomical order and stored at 4° C. in 24-well plates containing PBS with 0.06% Na-azide.

Results and Discussion

Overexpression of Bcl-2 in Bcl-2 Transfected NT2 Cells

The protein expression of Bcl-2 and its related proteins in NT2 cells was examined by Western blotting. The cells express a low but detectable level of Bcl-2 when they were cultured with the maintaining medium Opti-MEM 1 containing 5% FCS.

Whether Bcl-2 neurons express higher levels of Bcl-2 protein than NT2 neurons was then examined. A 0.91-kb cDNA clone of human Bcl-2 was inserted into the eukaryotic expression vector pcDNA3 and transfected into NT2 cells. The cloning efficiency of the Bcl-2 transfection, i.e. was higher than that of control transfection using the vector without the Bcl-2 cDNA insert (herein called neo), pcDNA3-neo. As shown in FIG. 1, the individual cell clones were screened for the expression of the Bcl-2 protein with Western blot analysis. Bcl-2-C5, Bcl-2 A5 and Bcl-2-A4 denote the isolated and successfully transfected clones. These clones all contain several hundred fold higher levels of the bcl-2-protein than untransfected NT2N neurons.

Neuronal Differentiation of NT2 and Bcl-2 Cells and the Expression of the Bcl-2

In the presence of retinoic acid (RA) for 5 weeks, both NT2 and Bcl-2 cells differentiate into a neuronal phenotype (which expresses neurone specific enolase, neurofilament subunits NFL (short subunit of neurofilaments) and phosphorylated NFH (heavy subunit of neurofilaments). Naive and neuronal differentiated NT2N or Bcl-2 cells were compared with respect to expression of Bcl-2 mRNA using RT-PCR and western blotting. Naive NT2 cells incubated under standard conditions expressed low levels of Bcl-2 mRNA (not shown) and of the Bcl-2 protein (FIG. 1). The Bcl-2 cells treated with RA showed up-regulation of the mRNA and of Bcl-2 protein. Five weeks after the RA treatment, many NT2 or Bcl-2 cells differentiated into neurons. Pure neuronal cultures were then obtained after replating and treatment with inhibitors of mitosis (see Methods).

Transplant Size and Survival

Sixteen days after transplantation of the Bcl-2 neurons to the striatum or hippocampus, the transplant had a maximal surface area of 1.1×10⁶ μm² meanwhile the NT2N neurons only occupied a maximal surface area of 0.65×10⁶ μm², i.e. 40% smaller (Table 1). At 60-78 days the Bcl-2 neurone transplants to the hippocampus and striatum decreased in size in comparison to that at 16 days by 20% of the original size. This means that approximately 100,000 cells had survived. NCAM immunoreactive neuronal cells and dense sprouting was measured. The ratio between cells and processes was not easily determined, i.e. a larger proportion sprouting at 60-78 days after transplantation would make the number of surviving neurons slightly smaller. At 240 days after transplantation of Bcl-2 neurons, the transplant size was similar to that determined at 16 days. This indicates that Bcl-2 neurons, which survive at 16 days after transplantation also, survive for 240 days. The maximal size of the Bcl-2 neuron transplants were 5 times larger than that of NT2N transplants at 16 days and 10 times as large at 240 days after transplantation. In 30% of the NT2N transplanted rats, no surviving human neurons were observed 60 days or later following transplantation.

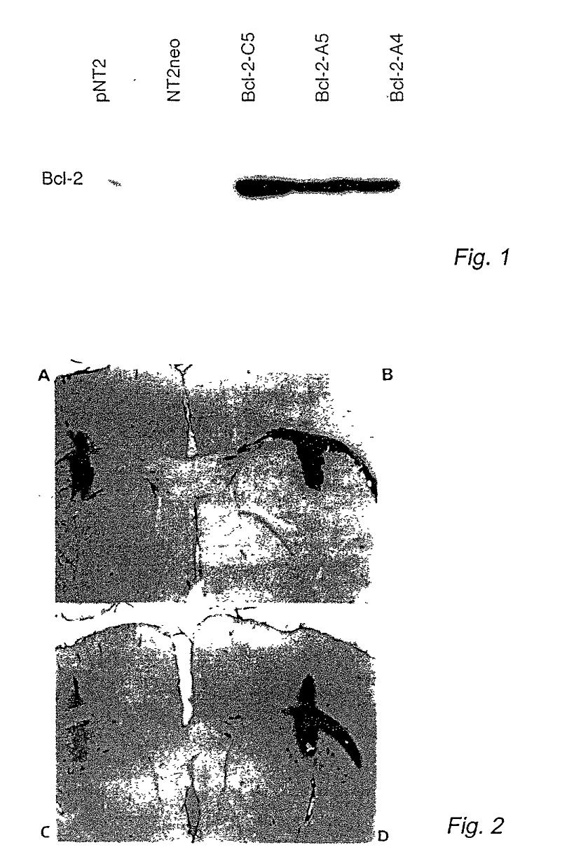

FIG. 2 shows the results of administration with a stereotactic device of 1×10⁵ NT2Nneo neurons (the two pictures to the left) or the same number of Bcl-2-neurons (clone Bcl-2-C5) (the two pictures to the right). The section with the largest area of the transplant from each rat is shown in the figure. Two weeks after transplantation, Bcl-2-neurons appear with distinctly longer sprouting processes (upper right picture) than does the non-transfected Bcl-2 NT2N neurons (upper left picture). Eight weeks after transplantation the area of Bcl-2-neurons is somewhat smaller (lower right picture) than at two weeks but the transplanted NT2N neurons have most disappeared.

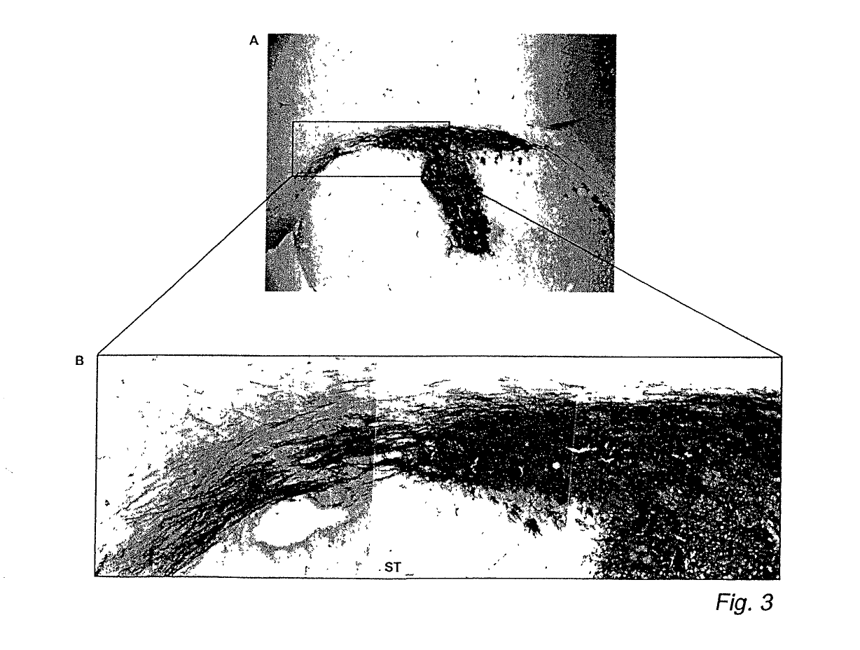

In Bcl-2 transplanted rats 16-30 days after transplantation, dense sprouting extended for 4-6 mm. However, processes of some Bcl-2 neurons could be traced for more than 1 cm. FIG. 3A shows that the neurons sprout at the border of striatum and in the white matter. Neurons also reach over the midline to the left. Sprouting occurs also into the striatum but to a much lower extent. The higher magnification of FIG. 3B shows delicate axons speeding from the bulk of sprouting axons both upwards and into the striatum. NT2N transplanted neurons were less successful and survived only in 3 out of 5 rats and in one of the rats the maximal length of dense sprouting was 2 mm.

TABLE 1
Survival and maximal surface area of transplanted Bcl-2
or NT2N neurons to striatum or hippocampus

| | Maximal | | | | |
|---|---|---|---|---|---|
| Days after trans- | surface area | | Maximal surface | | |
| plantation to hippo- | (μm²) × 10⁶ of | n = | area (μm²) × 10⁶ | | NT2N in |
| campus and | Bcl-2-neurons | successful/ | of NT2N- | n = | % of |
| striatum | (†SD) | failed | neurons (†SD) | successful/failed | (P>) |
| 16 | 1.088 | 9/9 | 0.649 | 7/8 | 60 |
| | (0.018) | | (0.032) | | (0.01) |
| 60-78 | 0.203 | 8/8 | 0.0449 | 5/5 | 22 |
| | (0.025) | | (0.040) | | (0.001) |
| 240 | 0.280 | 5/5 | 0.034 | 2/5 | 12 |
| | (0.012) | | | | (0.001) |

Transplantation and Immunosuppression

In order to determine whether immunosuppression with cyclosporin A could improve the survival of Bcl-2 or NT2N neurons after transplantation to the rat nervous system, 10 rats were given cyclosporin A by i.p. injections (1.5 mg/rat-250 g/day). Five rats were given Bcl-2 transplants and the other five rats NT2N transplants (1×10⁵ cells/transplantation) to the hippocampus. At 2 months the animals were investigated for human NCAM. As can be seen from Table 2 the maximal transplant area of 2 months old Bcl-2 neuronal transplants to the rat was similar to the area given in Table 1, while the size of the NT2N transplants were approximately 3.3 times smaller demontrating that the survival of NT2N neurons does not improve after immunosuppression. Furthermore, the difference in growth and survival between neuronal transplants of Bcl-2 and NT2N cells does not depend on the immunocompetence of the accepting animal.

TABLE 2
Survival and maximal surface area of Bcl-2- or NT2N-neurons trans-
planted to the rat nervous system in rats subjected to immunosuppression
with cyclosporin A (i.p. 0.5 mg/kg body weight/day)

| | Maximal surface | |
|---|---|---|
| | area (μm²) of Bcl- | Maximal surface |
| Days after trans- | 2 transplants | area (μm²) of NT2N |
| plantation | (†SD) | transplants (†SD) |
| 60 | 167000 (96000) | 49700 (33000) |

p > 0.01

Transfection Stability

The Bcl-2 neurons according to the present invention have been shown to be stably transfected for more than 30 months.

Transplantation of Bcl-2 Neurons to the Transected Rat Spinal Cord

Bcl-2 neurons transplanted to the transected rat spinal cord survived for at least 6 months. The spinal cord with the transplant (2.3-2.5 cm segments) was investigated in eight rats. As shown in the photomicrographs in FIG. 4, after 14 days in three out of eight segments of the rat spinal cord, the human Bcl-2 nerve cells regenerated through the whole examined part of spinal cord, both in proximal and distal directions. This is shown as black stain in the top figure. Slides 2 and 3 are from the same rat. In the microscopic photomicrographs (A-D), A shows human Bcl-2 neurons (black) with regenerating axons directed to the right. The white dots indicate where the photomicrographs were obtained. Axons regenerated in all segments, but to a greater extent in the proximal stump (A) as compared to the distal stump (C). Figure D is a magnification of the human neurons in figure B. Regeneration occurs mainly in white matter. The rate of regeneration was approximately 9-10 mm/per 10 days (FIG. 4). The reason for failure in 5 rats was organized bleedings, cyst formation, inflammatory granulation tissue and scar formation.

Bcl-2 transfected neurons transplanted to the transected rat spinal cord survive for at least 6 months. Of eight rats, three showed an advanced regeneration from the transection both distally and proximally (FIG. 4). The reason for failure in five rats was organized bleeding and scar tissue formation. In the successfully regenerating neurons the regeneration per day corresponds to the slow axonal transport of 1 mm per day, a speed at which neurofilaments are transported in the axon.

The results demonstrate that the upregulation of Bcl-2 provides for the long-term survival of human neurons upon in vivo transplantation to the central nervous system of mammals. Accordingly, the present invention provides extraordinary benefits to mammals suffering from neurological disorders in which transplantation and long-term survival of neurons may be beneficial.

### REFERENCES

Behl, C., L. Hovey, S. Krajewski, D. Schubert, J. C. Reed, Bcl-2 prevents killing of neuronal cells by glutamate but not by amyloid beta protein. Biochem. Biophys. Res. Commun., 1993, 197: 949-956.

Dubois-Dauphin, M., H. Frankowski, Y. Tsujimoto, J. Huarte, J. C. Martinou, Neonatal motoneurons overexpressing the bcl-2 protooncogene in transgenic mice are protected from axotomy-induced cell death, Proc. Natl. Acad. Sci. U.S.A., 1994, 91: 3309-3313.

Fung, K. et al. A novel modification of the Avidin-Biotin Complex, Method for immunohistochemical studies of transgenic mice with murine monoclonal antibodies, J. Histochem. Cytochem. 1992, 40:1319-1328.

Garicia, I., I. Martinou, Y. Tsujimoto, J. C. Martinou, Prevention of programmed cell death of sympathetic neurons by the bcl-2 proto-oncogene, Science, 1992, 258: 302-304.

Martinou, J., C., M. Dubois-Dauphin, J. K. Staple, I. Rodriguez, H. Frankowski, M. Missotten, P. Albertinin, D. Talabot, S. Catsicas, C. Pietra, J. Huarte, Overexpression of BCL-2 in transgenic mice protects neurons from naturally occurring cell death and experimental ischemia, Neuron, 1994, 13: 1017-1030.

Merry, D. E., D. J. Veis, W. F. Hickey, S. J. Korsmeyer bcl-2 protein expression is widespread in the developing nervous system and retained in the adult PNS, Development, 1994, 120: 301-311.

Michaelidis T M, Sendtner M, Cooper J D, Airaksinen M S, Holtmann B, Meyer M, Thooenen H., Inactivation of bcl-2 results in progressive degeneration of motoneurons, sympathetic and sensory neurons during early postnatal development. Neuron, 1996, 17:75-89.

Nakayama K I, Nakayama K, Negishi I, Kuida K, Shinkai Y, Louie M C, Fields L E, Lucas P J, Stewart V, Alt F W, Loh D. Y., Disappearance of the lymphoid system in Bcl-2 homozygous mutant chimeric mice. Science, 1993, 261: 1584-1588.

Oppenheim R. W., Cell death during development of the nervous system. Annu. Rev. Neurosci. 1991, 14:453-501. Pleasure, S. J., C. Page, V. M. Y. Lee, Pure, postmitotic, polarized human neurons derived from Ntera 2 cells provide a system for expressing exogenous proteins in terminally differentiated neurons, J, Neuronscie., 1992, 12: 1802-1815.

Reed J. C., Double identity for proteins of the Bcl-2 family. Nature, 1997, 387:773-776.

Renfranz, O. J. et al. Region-specific differentiation of the hippocampal stem cell line HiB5 upon implantation into the developing mammalian brain, Cell, 1991, 1566: 713-729.

Sambrook, J., E. F. Fritsch, T. Maniatis, Molecular Cloning: A Laboratory Manual. Cold Spring Harbor Press, New York, 1989.

Satou, T., B. J. Cummings, C. W. Cotman, Immunoreactivity for Bcl-2 protein within neurons in the Alzheimer's disease brain increases with disease severity. Brain Res., 1995, 697: 35-43.

Sawa, A., F. Oyama, N. J. Cairns, N. Amano, M. Matsushita, Aberrant expression of bcl-2 gene family in Down's syndrome brains, Mol. Brain. Res., 1997, 48: 53-59.

Veis D J, Sorenson C M, Shutter J R, Korsmeyer S J (1993) Bcl-2-deficient mice demonstrate fulminant lymphoid apoptosis, polycystic kidneys, and hypopigmented hair. Cell, 1993, 75:229-240.

Wiestler, et al., Retrovirus ñ mediated oncogene transfer into neural transplants, Brain Pathol., 1992, 2:47-59.

Zhong, L. T., T. Sarafian, D. J. Kane, A. C. Charles, S. P. Mah, R. H. Edwards, D. E. Bredesen, bcl-2 inhibits death of central neural cell induced by multiple agents, Proc. Natl. Acad. Sci. U.S.A., 1993, 90: 4533-4537.

1. A composition for the in vivo treatment of neurological conditions in mammals comprising Bcl-2-transfected neurons.

2. The composition of claim 1, wherein the neurons are NT2N neurons.

3. The composition of claim 2, wherein the neurons are deposited as DSM ACC2505.

4. The composition of claim 1 further comprising retinoic acid.

5. The composition of claim 1, wherein the neurological condition is selected from the group consisting of: Alzheimer's disease, Parkinson's disease, amyotrophic lateral sclerosis (ALS) and spinal trauma.

6. The composition of claim 1, wherein the neurological condition is Alzheimer's disease.

7. The composition of claim 1, wherein the neurological condition is Parkinson's disease.

8. The composition of claim 1, wherein the neurological condition is amyotrophic lateral sclerosis (ALS).

9. The composition of claim 1, wherein the neurological condition is the result of spinal trauma.

10. The composition of claim 1, wherein said composition is suitable for transplantation in mammals.

11. A method for the in vivo treatment of neurological conditions in mammals comprising administering Bcl-2-transfected neurons to a mammal in need of said treatment.

12. The method of claim 11, wherein the Bcl-2-transfected neurons are transplanted into the CNS or PNS of the mammal.

13. The method of claim 12, wherein the Bcl-2-transfected neurons are transplanted into the brain.

14. The method of claim 12, wherein the Bcl-2-transfected neurons are transplanted into the spinal cord.

15. The method of claim 11, wherein the neurons are NT2N neurons.

16. The method of claim 15, wherein the neurons are deposited as DSM ACC2505.

17. The method of claim 11, wherein the Bcl-2 transfected neurons further comprise retinoic acid.

18. The method of claim 11, wherein the neurological condition is selected from the group consisting of: Alzheimer's disease, Parkinson's disease, amyotrophic lateral sclerosis (ALS) and spinal trauma.

19. The method of claim 11, wherein the neurological condition is Alzheimer's disease.

20. The method of claim 11, wherein the neurological condition is Parkinson's disease.

21. The method of claim 11, wherein the neurological condition is amyotrophic lateral sclerosis (ALS).

22. The method of claim 11, wherein the neurological condition is the result of trauma to the spinal cord.

23. A composition for the in vivo treatment of a neurological condition comprising Bcl-2-transfected neurons, wherein said neurons produce dopamine upon transplantation into a mammal.

24. A method of treating Parkinson's disease in mammals comprising administering Bcl-2 transfected neurons to a mammal in need of such treatment.

25. The method of claim 24, wherein the Bcl-2 transfected neurons are administered by transplantation.

26. The method of claim 25, wherein the mammal is a human.
